(19) United States
(12) Patent Application Publication
(10) Pub. No.: US 2022/0030975 A1
(43) Pub. Date: Feb. 3, 2022

(54) PERSONAL PROTECTIVE EQUIPMENT AND METHODS FOR PREVENTING SPREAD OF INFECTIOUS DISEASE

(71) Applicant: Joseph P. Schultz, Atlanta, GA (US)

(72) Inventor: Joseph P. Schultz, Atlanta, GA (US)

(21) Appl. No.: 17/207,328

(22) Filed: Mar. 19, 2021

Related U.S. Application Data
(60) Provisional application No. 63/005,131, filed on Apr. 3, 2020.
(60) Provisional application No. 62/991,964, filed on Mar. 19, 2020.

(51) Int. Cl. A41D 13/11; A62B 23/02; A61M 16/06

(52) U.S. Cl. CPC A41D 13/1176; A61M 16/0688; A62B 23/025; A41D 13/1184

(57) ABSTRACT

Disclosed herein are personal protection equipment systems which provide improved protection from infectious agents, and can be constructed and distributed in high volume during public health crises, such as global pandemics where supplies become limited and bioterrorism events where public panic and uncertainty can provide additional obstacles to conventional protective measures. Methods of implementing protective equipment are also disclosed herein. The protective biohazard equipment disclosed herein can be made in a short amount of time at a low cost using readily available materials.

### CROSS-REFERENCE TO RELATED APPLICATIONS

This application claims the benefit of U.S. Provisional Patent Application No. 62/991,864, filed on Mar. 19, 2020, and U.S. Provisional Patent Application No. 63/005,131, filed on Apr. 3, 2020, the disclosures of which are incorporated herein by reference in their entirety.

### FIELD OF THE INVENTION

The present disclosure generally relates to personal protective equipment that can be readily produced and distributed during public health crises during interruptions to traditional systems and methods. The present disclosure relates to personal protective equipment which demonstrates better performance in the prevention of transmission of infectious disease through the adaptation of filters and respiratory equipment designed for a separate use. More particularly, the present disclosure relates to respiratory filtering devices having a filter component and a sealing component capable of reducing or even eliminating unfiltered airflow from reaching the user of the device.

### BACKGROUND OF THE INVENTION

During public health crises, the administration of medical services to the public is crucially important to mitigate adverse effects and prevent spread of infectious disease. The need for medical services often places healthcare workers at great risk of being exposed to infectious disease. Respiratory protection is often employed in an attempt to prevent transmission of aerosolized infectious particles. Ideally, systems which completely isolate a healthcare worker are employed to ensure the greatest protection.

For instance, in the recent COVID-19 pandemic, healthcare workers have employed HazMat suits while collecting test samples in drive-through booths in order to prevent exposure to the disease. However, HazMat suits can be cumbersome and interfere with more involved medical interventions and procedures, such as taking blood or inserting an IV catheter. Moreover, HazMat suits are not widely available in large numbers to address situations where the need for medical services overwhelms the capability of medical infrastructure, as occurs during pandemic conditions, and is the explicit goal of bioterrorism events.

In such situations, less effective substitutes are often used in necessity. For instance, disposable respirators such as N95 masks are commonly used for protection from biohazards, and are now widely used in the public to prevent transmission of COVID-19 during pandemic conditions. However, these disposable respirators are frequently used incorrectly due to design flaws. Particularly, respiratory masks require a fitted seal to prevent airflow from circumventing the filter, in order to be effective in preventing the transmission of aerosolized particles of an infectious agent. An unfitted mask may prevent a user from direct contact of the infectious agent, such as may occur by a patient's unrestricted cough inadvertently directed at another. Yet, unfitted masks offer little an illusion of protection against the transmission of aerosolized particles since the inhaled air will go preferentially around a filter and through a gap with a lower resistance. A person may put themselves in a dangerous situation thinking that they have the proper safety gear to protect themselves, only to be at more risk. For example they may spend a long amount of time in close proximity to a patient with a dangerous airborne illness without a mask, thinking that their respirator will filter out the dangerous pathogens to prevent them from being inhaled in large and small droplet forms directly into the airway. Still, disposable masks are used in mass quantities for their limited effect due to their comparative availability and ease of use with more effective measures such as the HazMat systems discussed above. However, during pandemic conditions, the availability to healthcare workers of even these devices with limited efficacy can be reduced or eliminated by a panicked population.

Having a novel system might permit health care workers to use existing equipment repurposed for new lifesaving protection to allow them to safely continue to perform their jobs with less fear of getting sick and less risk of getting sick. They would also have a more effective respirator system that would provide more than the just an illusion of safety. Thus, disposable personal protective equipment that can be readily constructed and distributed under pandemic conditions would be advantageous to prevent the effects of public panic. Disposable personal protective equipment with improved effectiveness against aerosolized particles and the transmission of infectious agents would also be advantageous to improve protection of healthcare workers and vulnerable populations.

### SUMMARY

Described herein are disposable personal protective equipment systems, and methods for constructing and using such systems for the prevention of transmitting infectious disease. Disposable respiratory systems with improved effectiveness against the transmission of aerosolized particles are disclosed herein. The systems and methods disclosed herein can be effective under pandemic conditions.

Facial tissues comprising an absorbent layer and an impermeable backing layer are disclosed herein.

### BRIEF DESCRIPTION OF THE FIGURES

The accompanying drawings, which are incorporated in and constitute a part of this disclosure, illustrate various embodiments of the present invention. In the drawings:

FIG. 1 depicts an embodiment of a face shield constructed from a rigid piece of plastic and eyeglasses.

FIG. 2 depicts an embodiment of a respiratory hood.

FIG. 3A depicts an embodiment of a roll of self-adhering plastic mask with an integrated filter as a continuous strip.

FIG. 3B depicts an embodiment of a roll of self-adhering plastic mask with an integrated filter in a repeating island filter pattern design.

FIG. 4 depicts the self-adhering plastic mask of FIG. 3 applied to protect the nose and mouth of a user.

FIG. 5 depicts a cushioned rigid frame connected to a filter to form an embodiment of a respiratory mask.

FIG. 6 depicts the frame and filter elements of FIG. 5 in a disconnected arrangement.

FIG. 7 depicts an embodiment of a mask construction system compatible with the rigid frame depicted in FIG. 5, with the filter being disposed within the ventilator connector of the rigid frame.

FIG. 8 depicts another embodiment of a mask construction system compatible with the rigid frame depicted in FIG. 5, designed to secure a filter on the outer surface of a ventilator connector of the rigid frame.

FIGS. 9A-9B depict another embodiment of a respirator mask.

FIG. 10 depicts an embodiment of a body shield.

FIG. 11 depicts several embodiments of protective boxes.

FIG. 12 depicts further embodiment of protective boxes and components thereof.

FIG. 13 is a photo of an embodiment of a face shield.

FIG. 14 depicts a gasket kit of the present invention.

FIG. 15 represents a perspective view of an embodiment of a gasket kit.

FIG. 16 represents a front view of a gasket kit shown in FIG. 15.

FIG. 17 represents a bottom view of a gasket kit shown in FIG. 15.

FIG. 18 represents a top view of a gasket kit shown in FIG. 15.

FIG. 19 represents a side view of a gasket kit shown in FIG. 15, the left and right side views being mirrored of each other.

FIG. 20 represents a perspective view of a gasket kit of the present design.

FIG. 21 represents a front view of a gasket kit of the present design.

FIG. 22 represents a bottom view of a gasket kit of the present design.

FIG. 23 represents a top view of a gasket kit of the present design.

FIG. 24 represents a side view of a gasket kit of the present design, the left and right side views being mirrored of each other.

FIG. 25 depicts a front view of an embodiment of a body shield disclosed herein.

FIG. 26 depicts a perspective view of the body shield shown in FIG. 25.

### DEFINITIONS

Equipment disclosed herein is generally described for the protection of individuals and bodily surfaces from contact with infectious agents, including aerosolized infectious agents. As used herein, the term ‘mask’ can refer to any arrangement of protective gear that protects a bodily surface from aerosolized infectious agents, including masks that cover the eyes, face, mouth, ears, nose, hair, arms, hands, etc., and any combination thereof. As used herein, respiratory masks refer to any arrangement of equipment that is designed to protect the respiratory pathway from contact with aerosolized infectious agents, and therefore can include protections that cover one or both of the nose and mouth. Respiratory masks as used herein can therefore encompass breathing masks that cover only the nasal openings and mouth, masks that extend from above the nose to the chin, respiratory hoods which cover the entire head, and full body protective gear.

### DETAILED DESCRIPTION OF THE INVENTION

Disclosed herein are personal protection equipment systems and methods of constructing and implementing said systems that result in an improved protection against the transmission of infectious disease. In certain embodiments, respiratory masks comprising a seal and a filter are contemplated. Certain respiratory masks may further comprise a rigid portion to allow a volume of internal airspace within the mask to improve. In other embodiments full respiratory hoods are contemplated having a volume of internal airspace, and can comprise a transparent household bag and integrated filter. Alternative embodiments may consist of a sealing material and an integrated filter within the sealing material, with effectively no volume of internal airspace to provide a secure seal and protect against unfiltered air.

### Respiratory Masks

Generally, respiratory (and non-respiratory) masks disclosed herein are disposable, and comprise a seal and a filter. Certain embodiments also can comprise a rigid frame to form an optional internal mask volume of filtered air. Respiratory masks disclosed herein generally also provide a sealing capable of effectively sequestering a user from unfiltered airflow entering the mask and exposing the user to infectious agents. The efficacy of these masks can approach or exceed that of relatively cumbersome, expensive, and inaccessible HazMat suits and hoods. Moreover, a seal between the user and the respiratory mask can be achieved without fitting procedures, as is currently required to achieve isolation from unfiltered air with rigid filtered masks suitable for mass production, including N95 masks. The principles and features of respiratory masks disclosed herein can also be applied to non-respiratory masks, which are also contemplated herein and discussed below.

Seals and sealing mechanisms of respiratory masks contemplated herein are not limited to any particular type, and generally can be any that are suitable to form a seal between the user and the mask, thereby preventing the user's airflow from incorporating unfiltered air. In certain embodiments, the seal, or sealing mechanism, can comprise a cushion, a gasket, a flap gasket, cream, ointment, glue, adhesive, or any combination thereof. In certain embodiments, the seal can comprise an air cushion, a foam cushion, closed cell foam cushion, a silicon cushion, or any combination thereof. In certain embodiments, the gasket can be formed from a rubber or plastic material, and may comprise any material suitable for forming the gasket. Again, any seal capable of, or configured to fit the contours of the face and form a complete seal are compatible with the respiratory masks disclosed herein, and contemplated by this disclosure.

Filters suitable for the embodiments contemplated herein are not limited to any particular material or form, and can generally be constructed of any material suitable to remove an infectious agent from the airflow entering the mask. For instance, in certain embodiments, the filter can comprise a conventional respiration mask, surgical mask, household dust mask, or any portion thereof. In alternative embodiments, the filter can comprise a facial tissue, an air filter adapted from an automobile or air conditioner filter, air filter adapted from a small stroke engine, vacuum bag, fibrous material, and any portions or combinations thereof.

In certain embodiments, the filter may have a porosity in any range suitable to prevent the contamination of an airflow with a particulate matter, e.g., an infectious agent. The infectious agents that can be filtered by embodiments contemplated herein are similarly not limited to any particular agent, and generally include bacterial and viral agents and their components in any form, e.g., aerosolized agents. Thus, in certain embodiments, filters contemplated herein can be any suitable to filter any amount of active or inactive infectious viral agents. Porosity of the filters contemplated herein can be in any range suitable to remove bacteria or viruses from the airstream. In certain embodiments, filters contemplated herein can have a porosity of less than about 100 microns, less than about 10 microns, less than about 5 microns, less than about 1 micron, less than about 0.5 microns, less than about 0.3 microns, less than about 0.2 microns, less than about 0.1 microns, less than about 0.05 microns, less than about 0.01 microns, or less than about 1 nm. Alternatively, the porosity of filters and filter materials contemplated herein can be in a range from about 0.01 microns to about 10 microns, from about 0.01 microns to about 0.5 microns, or from about 0.1 microns to about 1 micron.

Surprisingly, by combining the disposable filters with an effective seal, the respiratory masks (and non-respiratory masks) disclosed herein can remove a larger portion of an infectious agent from the airflow entering a user's respiratory pathway than a respiratory mask with a lower porosity. For instance, masks with a porosity of 0.3 microns and a sealing mechanism as described herein, can be more effective in reducing exposure to particulate matter of 0.1 microns or less than conventional disposable filters rated with a porosity of 0.1 microns.

The manner in which the components of the masks disclosed herein (e.g., the seals and filters discussed above) are combined is not limited to any particular construction or arrangement, and generally can be any suitable to seal and restrict unfiltered airflow from entering the sealed portion of the mask.

For instance, in certain embodiments, any seal as described above can be applied to a rigid frame commonly employed for any purpose, and unrestricted to by rigid frames associated with respiratory purposes. Any rigid frame or mask capable of surrounding the nose and mouth of the user and providing a seal against airflow can be suitable for the respiratory masks contemplated herein. Thus, in certain embodiments, the respiratory mask can further comprise a rigid frame or rigid mask such as an N95 mask, a CPAP mask, bipap mask, or bvm mask, and allow such devices to be used for purposes disclosed herein that are not envisioned by their originally intended use. In certain embodiments, such rigid masks can be sealed by applying the seals described above to the typically unsealed portion of the rigid mask that interacts with a user's face or body.

Respiratory masks comprising a filter attached to a rigid frame are also contemplated herein. In certain embodiments, a rigid full face mask for bipap could be part of a respiratory mask that comprises, consists essentially of, or consists of a respiratory filter cartridge at a standard opening of a mask with a standardized respiratory adapter. The respiratory mask contemplated herein may be unattached to a ventilator or anesthesia bag, and in contrary to typical practice where the mask is in gaseous communication and connected with a gas source. It is typically undesirable to have patients on these devices without ventilation support since the masks are thereby rendered inoperable for use in assisting ventilation.

Rigid constructions are also advantageous in extending the use of disposable respiratory masks. For instance, portions of the respirator could be wiped off without affecting the filtration. Additionally, the respiratory mask may be shared among many users, by disinfecting the rigid portion, replacing the filter, and applying a seal. In certain embodiments, the seal can be adaptable to many different facial structures such that a secure fit is achieved for any number of users, in a one-size fits-all construction with no need to fit a particular user to the respiratory mask.

Respiratory masks that combine a seal as contemplated above, with a rigid mask that may be transparent would be desirable at least because the rigid portion could be used to apply pressure upon the seal item. Such embodiments also allow existing medical components to be used for a new life saving purpose, which can become advantageous during periods of significant shortage of personal protective equipment, for example during global pandemics and other widespread health crises that a public panic response.

Masks that do not comprise a rigid frame are also contemplated herein. In such embodiments, respiration masks disclosed herein may consist of a seal and an integrated filter. In certain embodiments, a filter, or any filtering material disclosed herein may be attached to a flexible impermeable material such as a self-adhering plastic (e.g., Saran Wrap®). In such embodiments, the plastic may serve as the seal, or sealing mechanism as disclosed above, and be coupled directly to the filtering material. The self-adherent material can contain an integrated filter that allows air flow to be generated across the material while forming an excellent seal comparable to that achieved by HazMat suits. In such embodiments, the impermeable plastic can be produced as a roll of material with integrated filter. In certain embodiments, the roll may comprise a continuous strip of any filter material within the plastic material adjoining or in the middle of two strips of plastic material. Alternatively, the filter may be arranged in an interrupted array of filters spaced with gaps or windows of the plastic. In certain embodiments the plastic may be impermeable. Alternatively, the plastic may be semi-permeable, allowing only particles within a certain threshold to pass through.

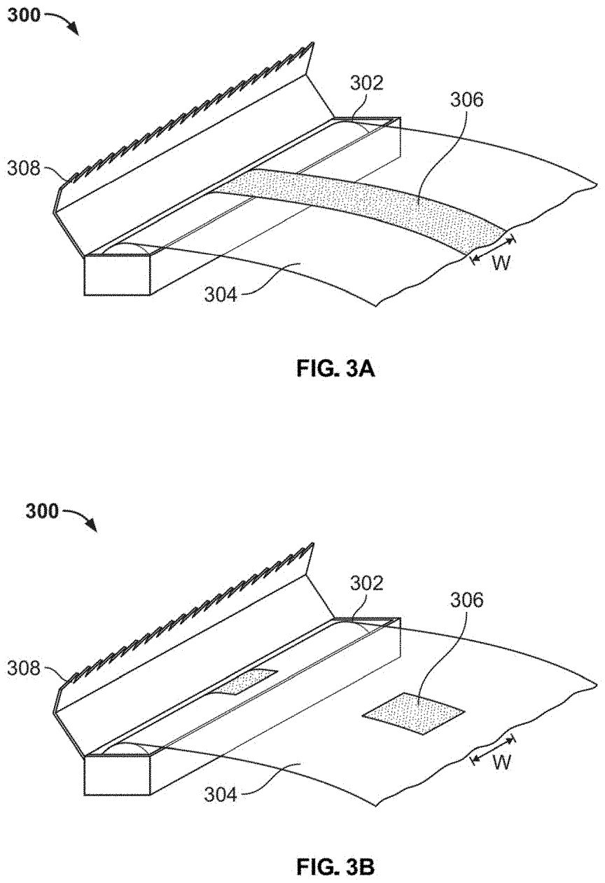

As shown in FIG. 3, the respiratory mask system 300 can comprise a standard self-adhesive plastic roll 302 packaged with in a box having serrated strip 308 to allow selection of an appropriate length according to the required size of the mask. Roll 302 comprises a filtering material 306 secured within plastic material 304 at an appropriate position. Filtering material 306 can be any appropriate filter and any filter disclosed herein compatible with the intended use. The filtering material 306 can have any width suitable to provide airflow to the nose, mouth, or both, during use of the mask. The filtering material 306 can be centered within the roll of plastic material, or offset to either side, and to any degree that allows a minimum sealing distance on each side of filter material 306. Additional arrangements of the filtering material are also contemplated herein.

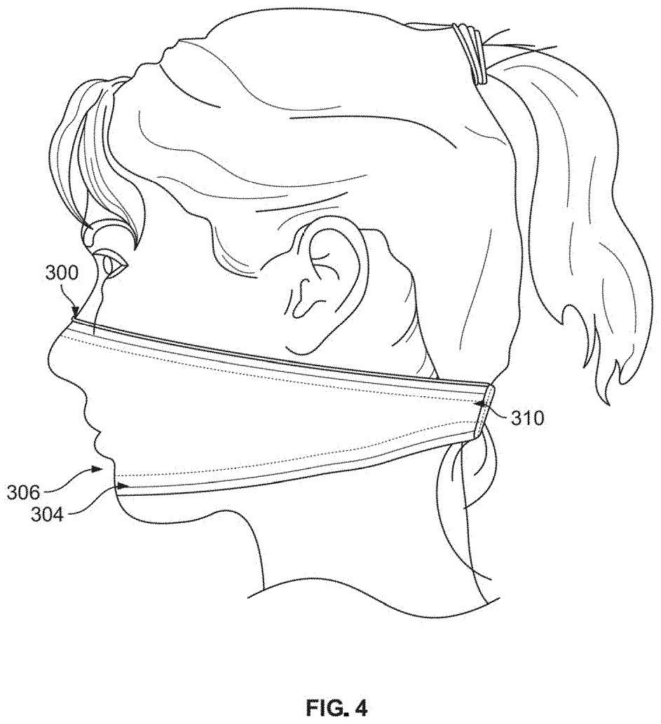

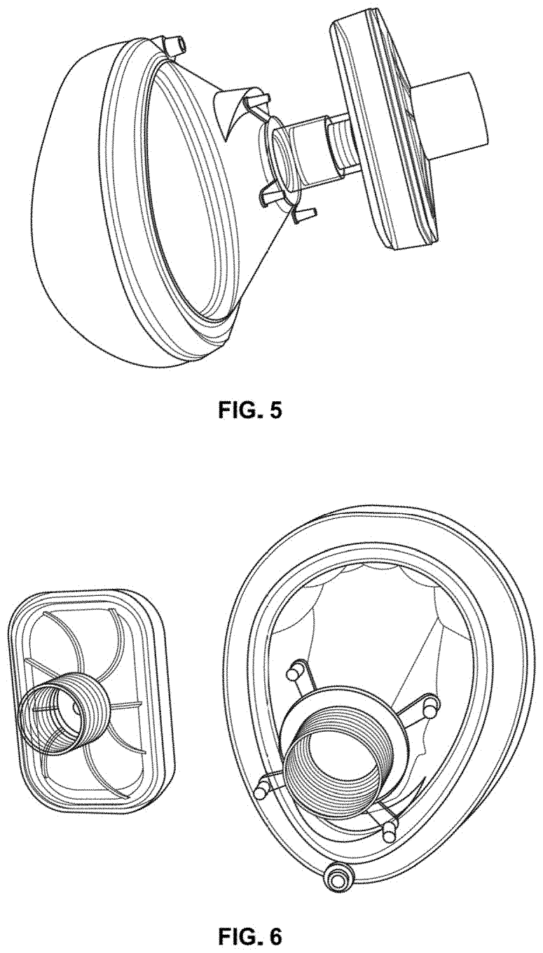

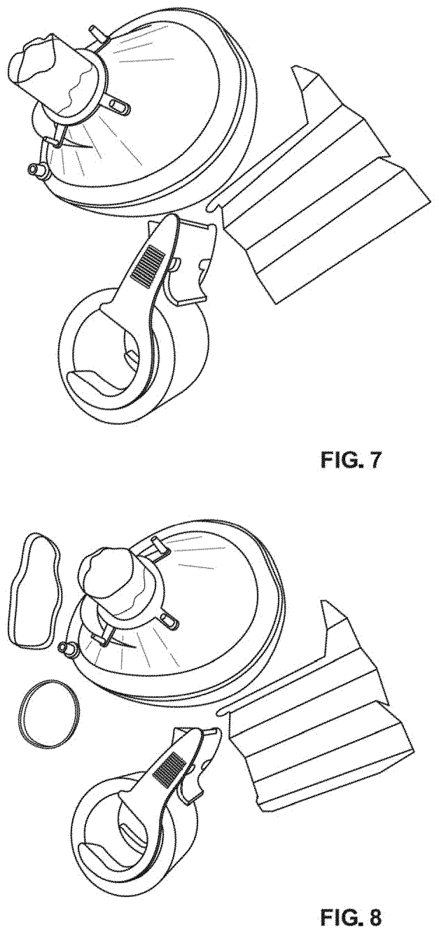

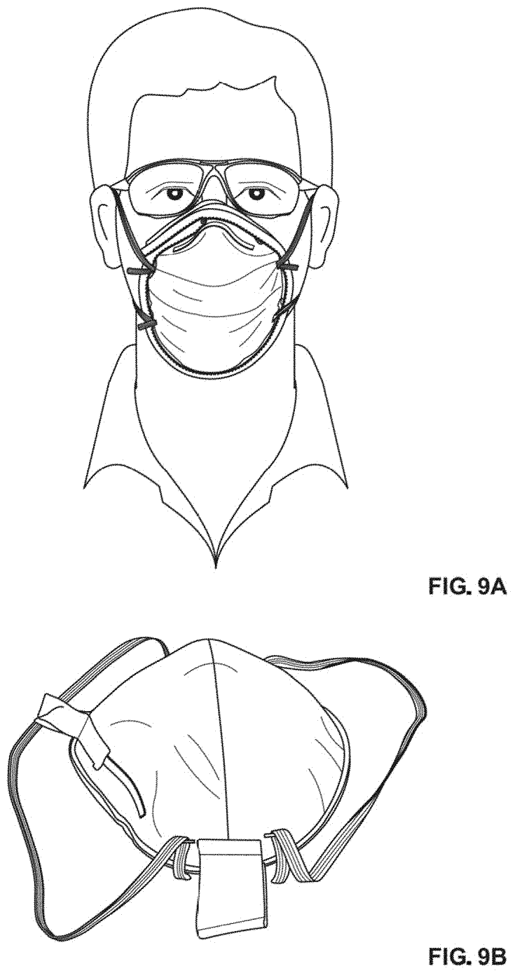

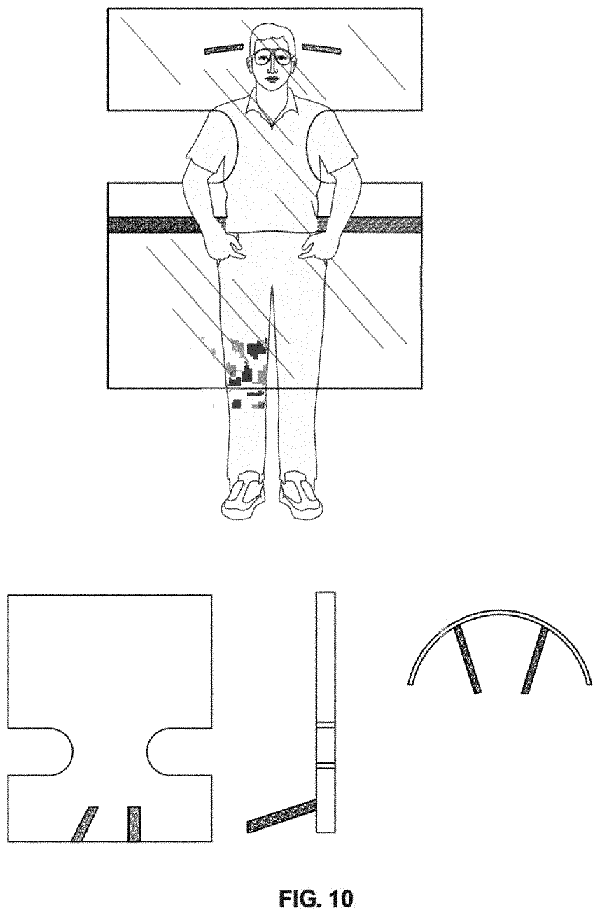

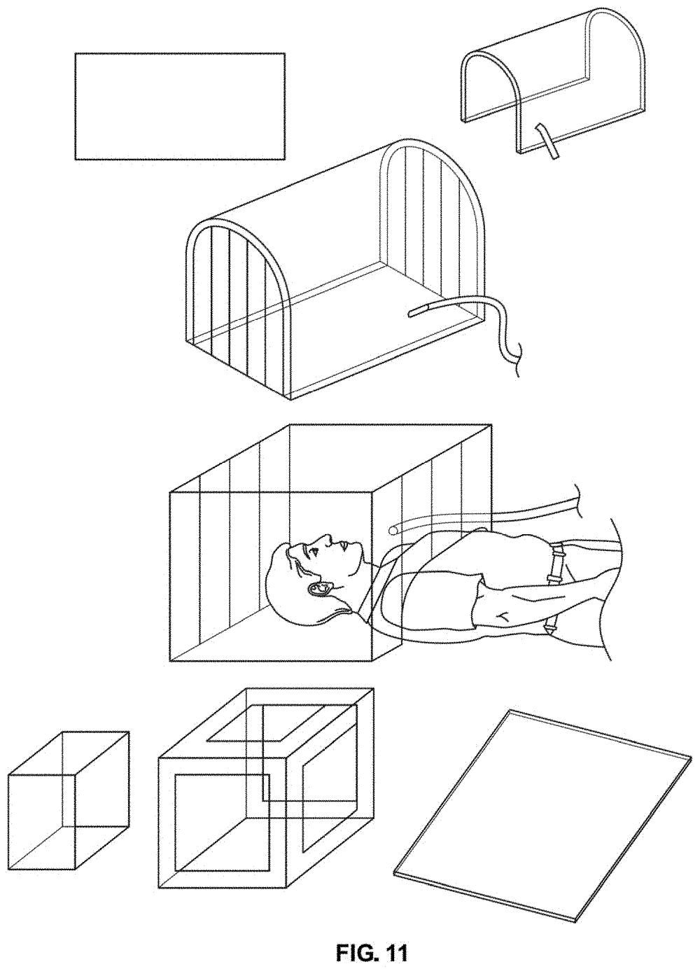

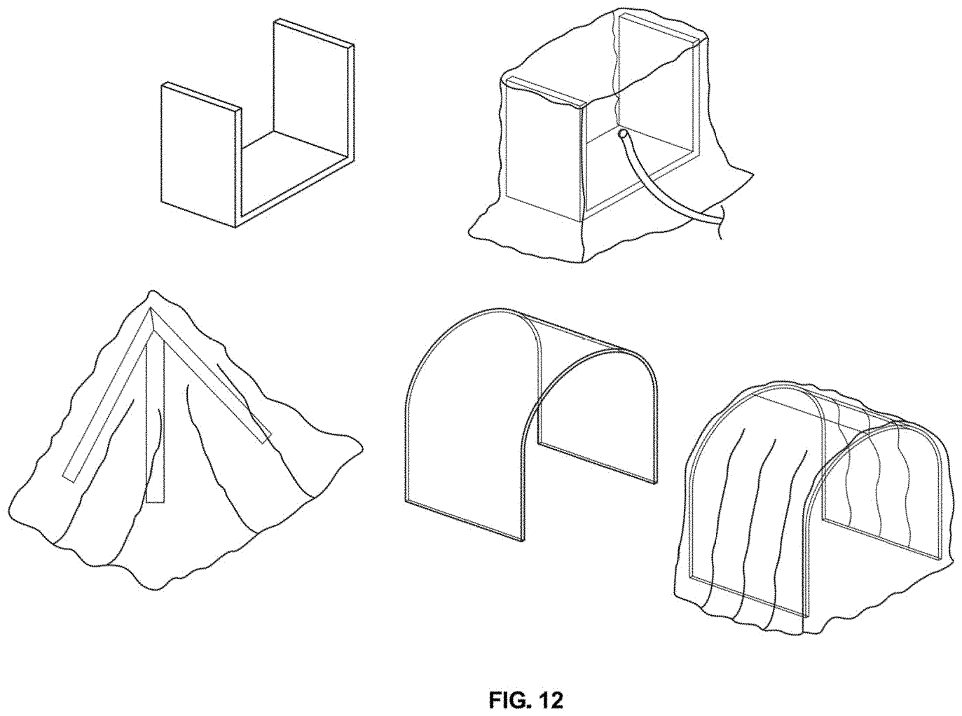

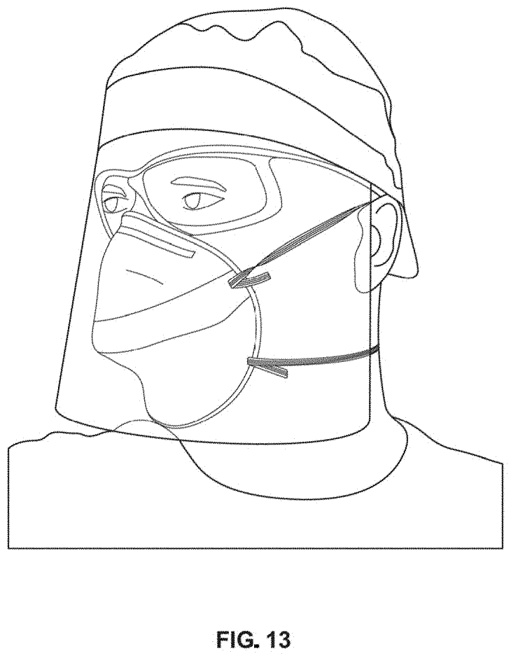

FIG. 4 depicts an application of mask 300, wherein filter material 306 covers the nose and mouth of the user, with self-adhering sealing plastic 304 prevents unfiltered airflow from entering the mask. Mask 300 can be secured to the user behind the neck at an overlapped portion of the mask 310, and provide pressure against the seal.

Embodiments of the invention can also be constructed using an impermeable plastic such as a large transparent bag or a strip of food wrapping and adding a filtering material such as an air conditioning filter paper that is sealed to the bag or plastic roll.

With respect to the arrangement of the filter, the filter can comprise a filter material inserted within inhalation and/or exhalation port or ports of a rigid frame. Alternatively, the filter may be integrated within a rigid frame by clamping or securing a filter to the frame in an airtight seal upon a frame or between two frames so there is no leak around the filter material forcing air to flow through the filter material. Certain embodiments may comprise a strap or straps secured to the rigid frame to secure the mask to the face covering the mouth, the nose, the eyes or any combinations thereof. The filter may also comprise a prefilter for filtering larger particles than the second filter, and preserving the utility of the filter. The filter may have an inlet or outlet extension. Certain aspects contemplated herein can comprise one-way valves to ensure proper flow of clean and exhaled air.

The filter may also be separately disposable and exchangeable to improve safety and further preserve stock of available equipment. For example, the filter can be exchanged when a filter gets dirty, clogged, overused or becomes a risk for transmitting microbial contamination. In certain embodiments, the filter may be encased in a cartridge, and the cartridge may be removable, transparent, flat, wide, contoured for increasing surface area, e.g. with folds. Filters formed as plates comprising surrounding impermeable surfaces are contemplated and may provide better adherence to the more flexible portions of a rigid or flexible frame, e.g. a bag or plastic wrap.

The masks contemplated herein can further comprise an active pump configured to pull air through the filter. The active pump can be battery operated or operated by a phone battery supply, and may be configured as part of the filter component itself. Certain embodiments can comprise an air pump such as a fan that may pull air through a filter and into an internal volume of the respiratory mask (e.g., a rigid mask or portable hood) to provide one-way flow of filtered air through the filter and out of the internal volume, making the filtered air more accessible to the user. Embodiments comprising active pumps as described may serve to assist a user's respiratory capacity in drawing air through the filter without compromising the purity of the internal volume of filtered airspace within the mask. Thus, the user would be able to breathe clean purified air within the internal volume, and provided by the filtered air source, at a rate greater than the user's tidal volume to ensure that dirty air is not pulled into the internal mask volume, and subsequently inhaled into the user's lungs. Passive pump mechanisms may also achieve a similar result, and are also contemplated herein.

Embodiments of respiratory masks contemplated herein may or may not comprise an internal volume within the mask isolated from unfiltered air. Examples of embodiments with an internal volume of filtered air can include those with rigid frames provided by CPAP masks and the like, as contemplated above, as well as embodiments of portable respiratory hoods and face shields having a significantly larger internal volume within the sealed portion of the mask. Alternatively, embodiments consisting of a seal and filter may be sealed directly to the user and have only a minimal internal volume within the sealed portion the mask, and adhere directly against the user's skin.

Face shields may also be constructed from household items by securing a transparent plastic material, such as a rigid plastic sheet, to a hat or cap (e.g., a billed baseball hat). In certain embodiments, the transparent shield can be attached at the base of a billed baseball cap, and the cap worn in reverse to allow the transparent shield to adapt to the form of the wearer's face. In such arrangement, the shape of the transparent shield will be curved providing smooth corners and a stronger arrangement. Any number, shape, and material of spacers may be employed to distance the plastic sheet from the wearer's face appropriately, and provide a comfortable seal between the shield and the hat. The shield may be attached to the hat by any suitable means, which in certain embodiments can comprise straps (e.g., elastic straps), tape about the cap circumference (e.g. duct tape).

In addition to providing benefits to the seal of an unfitted respiratory masks, embodiments and methods contemplated herein may also assist and improve the durability and reusability of respiratory masks, and prolong the useful life of conventional respiratory masks. Circumstances such as a pandemic crisis where ay healthcare personnel and resource stockpiles can be overwhelmed by the pressing need may require that healthcare personnel employ respiratory masks beyond their intended and typical uses as a single-use mask not configured for extended use throughout a single shift.

Conventional single use masks carry significant disadvantages and risks when employed for prolonged, multiple use setting. For instance, prolonged use of conventional masks can lead to an increased risk of contamination. Contacting the sides of the filter during adjustments and use can lead to contamination and ultimately infection where masks are not replaced regularly. Filter sides also can be degraded by chemicals leading to a weak point in filter and endanger personnel to exposure to infectious agents.

Embodiments disclosed herein provide a filter edge that can be easily handled and cleaned without damaging filter. In certain embodiments, the edge can comprise a seal such as weather stripping and provide an improved spongy cushioned seal. Cushioned seal can allow the mask to conform to the face and the mask, better providing restriction of unfiltered air from entering the internal volume of the masks. Such arrangements also prevent the illusion of safety from conventional single use masks. Cushioned seals can also adjust to the movement of the face and changes in contours that develop during various activities, and can generally be adjustable. When mask is a “mis-fit” due to stock limitation, seals contemplated herein can help with converting a conventional mask to a reusable or prolonged use mask.

In a similar manner to how poorly fitting masks against the face can allow unfiltered air to enter the internal volume of the mask, poorly fitting seals against the mask also allow for an opportunity for the mask structure to be degraded or incomplete and allow unfiltered air to enter the internal volume. Gaskets are contemplated herein to address this issue, and secure the edge seal to the mask in an air-tight manner.

Methods of attaching an edge seal to a conventional face mask are contemplated herein. The edge seal may be attached directly to the mask, or by the addition of an intermediate gasket to provide a seal as discussed above. In certain embodiments, the gasket can be prepared by aligning a gasket material with the mask and cutting an outline of the mask, cutting an internal circuit to provide the gasket. Methods contemplated herein can further comprise inserting the gasket within the edge seal and affixing the seal to the face mask edge.

The gaskets contemplated herein are not limited to any particular material, and generally may be any material capable of conforming between the mask edge and the edge seal to reduce the amount of unfiltered air flow entering the internal mask volume, provide a more rigid, or durable structure to the mask at the seal. Gaskets contemplated herein can comprise a cushioned material (e.g., an air cushion, a gel cushion). In other embodiments, gaskets contemplated herein can be created at home on demand, from a sheet of material as described below. In certain embodiments, the gasket may be selected from papers, metals, tapes (e.g., athletic tape), silicon materials, rubbers, plastics such as window cling, foams (e.g., closed cell foams, memory foams), an combinations thereof. The gasket may be a compressible material to allow pressure applied from the mask material against the face to induce a force against both the gasket and the seal, reducing the potential for gaps to form at the interface formed at the mask, gasket, seal, and skin.

In certain embodiments the gasket can comprise a uniform thickness throughout the material, or alternatively a non-uniform thickness to facilitate a seal. Gaskets may be produced from materials comprising markers (e.g., points of changes in thickness) to provide cutting guides for inner and outer rims of the gasket. Concentric markers may also be provided to guide the preparation of gaskets of different widths and sizes.

In certain embodiments of respiratory masks contemplated herein, any of the edge seals, single use masks, and gaskets can comprise a viscous material to adhere the mask, seal, or gasket to the user, or to another component of the mask. Viscous materials contemplated herein can comprise a slime, sealant, putty (e.g. Aaron's Thinking Putty). In certain embodiments, the viscous material can be demonstrate the ability to removably adhere to a surface and remain intact.

Gasket kits are also contemplated herein, and generally can be employed to convert any sort of material or mask into a secure mask for filtering particles. In certain embodiments, gasket kits contemplated herein can provide a seal between a user's skin and any manner of material disclosed herein, or commonly used as a facial covering, or for the filtering of air borne particles. Gasket kits can comprise, consist essentially of, or consist of a gasket and a seal.

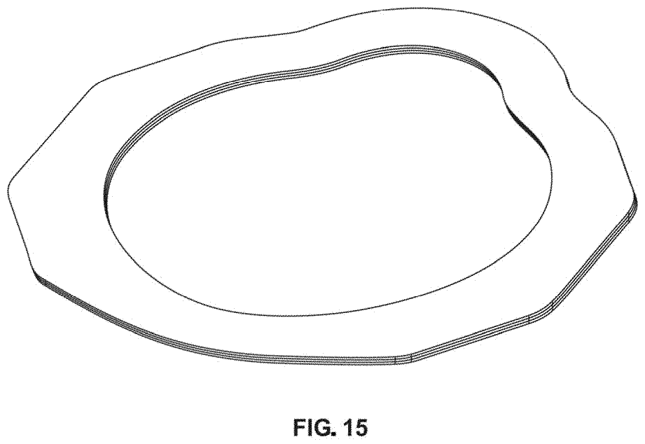

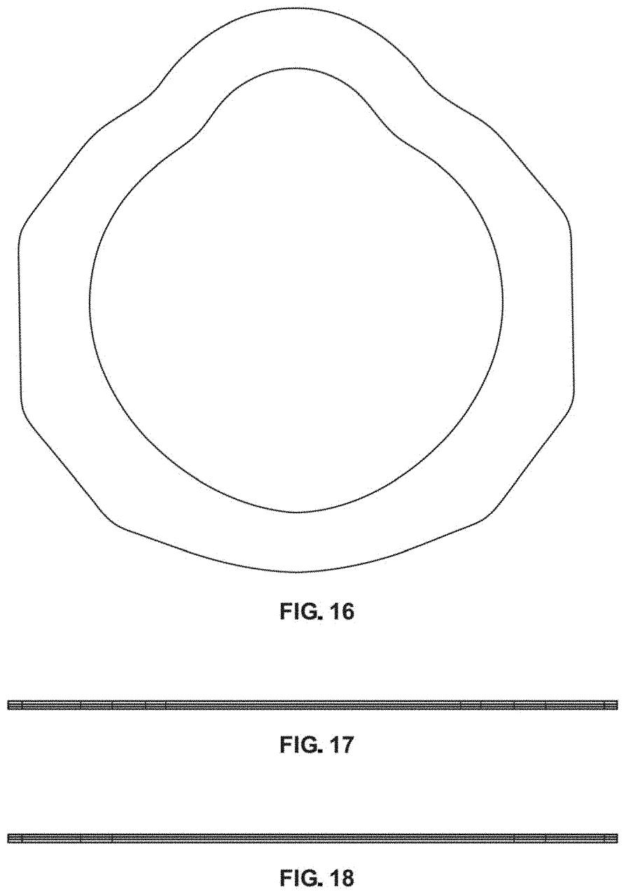

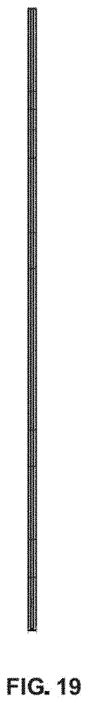

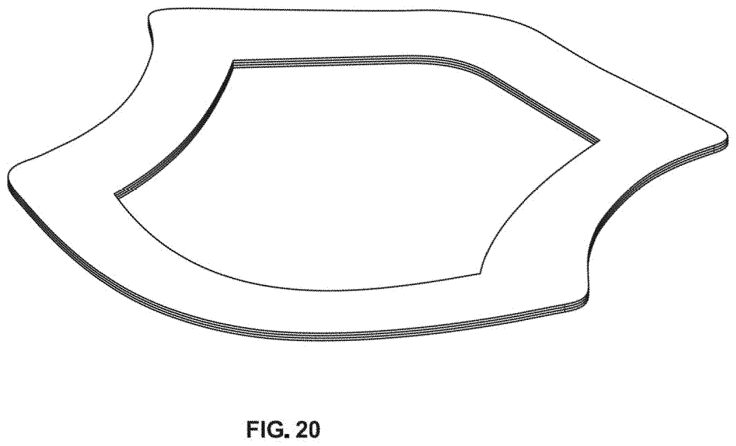

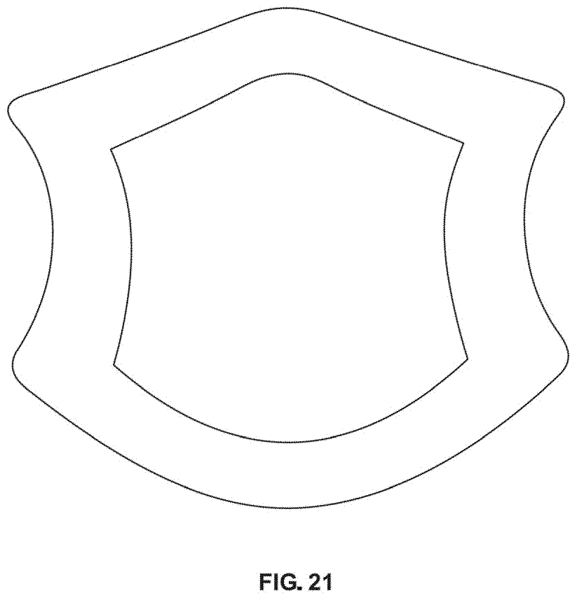

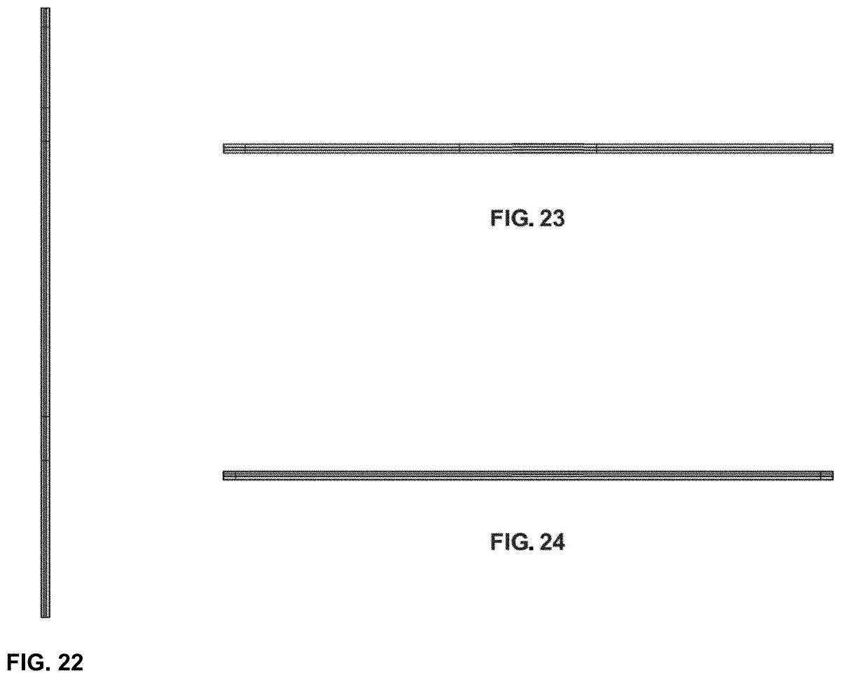

In certain embodiments, the gasket can comprise a cushioned, compressible gasket which can provide comfort to the user as well as conform to the contours of the face and secure the seal. Similarly, the gasket can comprise a sheet of any suitable material disclosed herein, with a seal incorporated into the gasket as a single unitary sheet. In such embodiments, the gasket kit can be disposable, and replaced against a reusable mask or mask material such that a custom fitted mask may be reused with the disposable gaskets. Gaskets may comprise a silicon edge, or be incorporated into a mask material as a silicon edge folded inward toward the interior portion of the mask providing a position for the seal to be applied. The gasket may also comprise a flared border such as can be seen on a coffee filter, or slits in the border to allow lateral overlapping of the gasket. Such aspects can be seen particularly at regions of the gasket subjected to the greatest shape contours of the face, including the transition between the bridge of the nose and the cheek, and the transition between the jaw line and the neck. Non limiting examples of gasket kits are shown in FIGS. 15-24.

As discussed above, the seal may be a gel, liquid, viscous oil or conformable solid material (e.g., putty, clay) that conforms to both the user's skin and the gasket, and provides an air seal between the user's skin, gasket, and the mask material. In certain embodiments, the gasket can be integrated within the seal such that the gasket provides a relatively rigid structure to the seal, while the seal extends to each surface of the gasket to provide a seal between the user's skin and the gasket, and also between the gasket and the mask material.

Seals contemplated herein may also have an adhesive quality, and in some cases serve to replace the elastic bands found on traditional masks to secure the mask against the face. It is contemplated that providing a more complete seal through the gasket and seals provided herein may reduce the amount of pressure needed to properly secure the mask to the face. Accordingly, certain embodiments of masks, gaskets, seals, and methods for using contemplated herein can comprise a seal, gasket, and mask, configured to be secured to the user's face with a minimal amount of applied pressure, or no pressure, such pressure typically being supplied by an elastic band around the head or ears. In said embodiments, traditional methods of securing the mask (e.g., elastic bands, cloth ties, etc.) which can often be the greatest point of discomfort, can be omitted from the mask design entirely. Sealing adhesive can be applied on a single side of the gasket, or on both sides of a gasket to secure the mask material to the gasket.

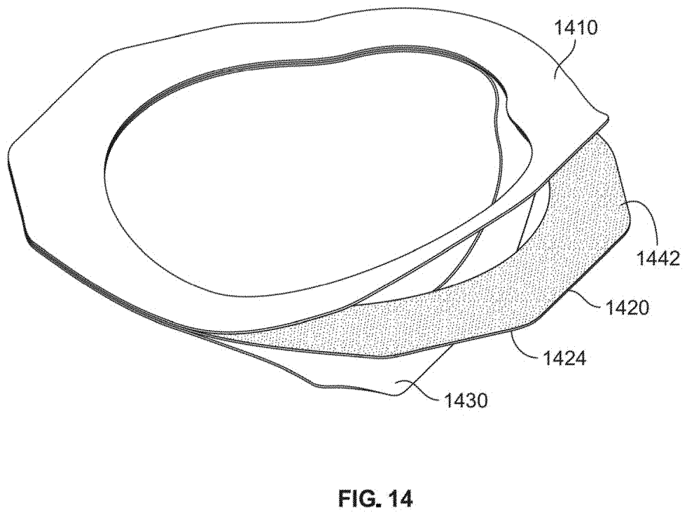

In certain embodiments, seals contemplated herein can comprise, consist of, or consist essentially of, an adhesive. The nature of the adhesive is not particularly limited, and can be any suitable to prevent or reduce airflow between any mask described herein and the user's skin. Alternatively, the gasket may have dual seals to provide a seal on each side of the gasket, as is shown by the example in FIG. 14. In such an arrangement, the adhesive may be strong enough to supplement the seal conventionally applied by pressure against the straps of the mask, or obviate the need for straps altogether in strapless mask embodiments. The gaskets and seals contemplated herein can be built into masks.

Adhesives can be arranged on the gasket in any manner, or can be applied separately from the gasket. In certain embodiments, the adhesive can add to the width of the gasket, such as where the gasket and seal are intended to form a compressible barrier. The adhesive may be repositionable to allow the mask to be adjusted throughout use and wear. In certain embodiments, the adhesive may be reactivated, and/or reapplied to regenerate the sealant properties of the adhesive. The adhesive may extend only a narrow portion of the gasket, either from the edge or the center of the gasket. The adhesive can comprise an oil, gel, such as lip balm or glue stick, and can be pre-applied, or applied onto the gasket by the user as a separate and independent component of the gasket sealing kits disclosed herein. In such aspects, the user may have control to reapply or augment the adhesive as necessary during use.

Gaskets may comprise rigid paper products such as cardboard, paper board, closed cell foams, tubing, EPDM rubbers, silicone sheets, static gels, inflatable tubing.

Particularly, FIG. 14 shows gasket kit 1400 having removable liners 1410 and 1430 attached in protection of adhesive layers 1422 and 1424 present on gasket 1420. The adhesive layers 1422 and 1424 can be any adhesive suitable for the purpose of securing the mask to the face, and restricting airflow. Once liners 1410 and 1430 are peeled away, the adhesive is exposed to allow securing any mask disclosed herein to the user's face, thereby converting said mask to a meet more stringent protection standards by reducing airflow around the edges of the mask. Liners 1410 and 1430 may be made of the same or different material. In certain embodiments, a skin-facing liner can be thin plastic only protecting the adhesive layer from being degraded during production, shipping, and handling prior to use. The mask-facing liner may comprise a rigid member to facilitate application of the gasket to the skin after removal of the skin facing liner. In certain aspects, the mask-facing liner can comprise a thick paper, such as cardboard, to provide structure to the gasket during application.

Methods of sealing a mask to a face are also disclosed herein, and can comprise peeling a first liner away from a first side of a gasket to expose a first adhesive layer, applying the exposed gasket layer on a portion of skin, peeling a second liner away from a second side of the gasket to reveal a second adhesive layer, and securing the mask to the gasket. Methods contemplated herein can be performed in any order. In certain aspects, the gasket may be pre-applied to a strapless mask or filtering material, the mask having straps or being strapless.

Methods to employ gasket kits are also contemplated herein, and can comprise applying the seal to the gasket, applying the seal to a mask material, and applying the gasket against the skin. The seal may be applied to the gasket on any or all portions. For instance, the seal can be applied to a single side of the gasket in between the mask and the gasket. Alternatively, or additionally, the seal can be applied between on the opposite side of the gasket meant to interface with the user's skin. In such embodiments where the seal is applied only between the gasket and user's skin, the gasket may be resistant to degradation by the seal material, thereby allowing the gasket to be cleaned for reuse while preventing the mask material from being degraded.

In certain embodiments, the mask material may be any mask or mask material contemplated herein. In certain aspects, the mask material may be transparent to allow others to observe facial cues and expressions of the wearer without compromising the filtering capacity of the mask. Such masks may have filtering material positioned to the side of the masks as opposed to covering the mouth, to allow the expressions to be seen clearly.

For instance, the gasket and seal may be applied to an N95 mask to better conform to the shape of the wearer's face and prevent gaps. The gasket kits may also be applied as described above to improve the filtering of KN95 masks according to Chinese regulatory standards, or masks prepared according to the regulations of any other governing body for the purpose of filtering air borne particles. Gasket kits may be applied to any sort of mask or mask material, including but not limited to respiratory masks, medical and surgical masks, dust masks, half face respiratory masks. Alternatively, the methods disclosed herein may be effectively applied to mask materials themselves, not commonly employed in masks or for the filtering of air borne particles. As such, the gasket kits and methods disclosed herein can provide effective filtration of air borne particles without dependence on a preformed mask, or specialized mask material.

N95 masks for instance can be formed from melt-blown polypropylene, in part to retain their three-dimensional shape. This three dimensional shape of N95 masks can be important to assist the edge seal during movement, and provide an ample surface area to allow filtered air to pass through the filter material as opposed to the gaps present between the skin and the mask edge. Methods contemplated herein can serve to minimize those gaps regardless of the shape of the mask or mask material, thereby mitigating the importance of the three dimensional shape of the mask in preventing airflow around the mask material, as opposed to through the material. The importance of rigidity provided by the melt blown polypropylene is also mitigated by gasket kits and methods disclosed herein, such that softer, more flexible, and ultimately, more comfortable materials can be employed for the purpose of filtering airborne particles.

Removable 3d shells are also contemplated herein in combination with gaskets and gasket kits of the present invention, such that the 3D shell may be reused even where the gasket kit may be disposable. In this manner, the use of three dimensional shaped mask materials may be prolonged such that shortages to supplies can be avoided or reduced in the context of widespread global need. In certain embodiments, the removable shell can removably attach to the gasket or gasket kit by means of a cassette clamp. In other embodiments, the removable shell can attach to an air cushion gasket.

Incorporation of a wider range of mask materials may allow more adaptable shapes for individual users. For instance, gasket kits can provide gaskets in the shape of a traditional, avocado-shaped profile, or any other profile that may be suitable for the user. The application of the gasket kits disclosed herein is not limited to hard shell or soft shell masks, and may also be applied to non-shell, and/or flat mask materials (e.g., surgical masks). Gasket kits can be applied to virtually any flexible mask material while retaining a similar, or improved level of filtration due to the seal at the edge. For instance, gaskets and gasket kits as provided herein can be custom fit to a user's facial hair, or preformed and manufactured according to common styles. In other embodiments, the gaskets can be formed to accommodate goggles, glasses, lenses, and the like. Such flexible, and non-shell components also have distinct advantages in manufacturing, cost, and ability to assemble with gasket kits for reusability as a removable liner.

Improvements to the sealing of common masks and mask materials provided by the gaskets, seals, gasket kits, and methods disclosed above allow further advantages. For instance, with less dependence on the mask material being a relatively rigid melt blown polypropylene, the mask materials may be adapted to include further specialized components directed to the removal of certain particles from the air. N95 masks for instance, were originally certified for the filtration of asbestos particles from the air, and repurposed en masse for use against the passage of microbial agents. However, the N95 certification was not targeted as demonstrating an effectiveness for filtration of viral particles, which are much smaller in size relative to the particles for which the certification was designed.

In this consideration, it is contemplated that hydrophilic and/or hydrophobic mask layers may prevent biologic particles from traversing the mask material more efficiently, as such particles commonly carry external facing proteins depending on their environment. It is contemplated herein that including hydrophobic layers within the mask structure may repel unwanted particles, or cause such particles to accumulate outside the internal airspace of the mask. Again, it is also contemplated that hydrophobic and hydrophilic layers may be employed depending on the electrostatic nature of the particle to be filtered.

Masks are also contemplated having charged layers to repel, attract and retain, or both unwanted particles from entering the internal air volume of the mask. For instance, negatively and positively charged layers may be created across the mask material in the same manner as a capacitor, to actively filter charged particles from the air that passes through the mask material. Charged layers may be incorporated into the design of the mask by any practical manner. For instance, masks contemplated herein may comprise a power source. The power source can be an integral battery. Alternatively, a separate power source may be supplied and connected to the mask to provide an increased amount of power or improve compatibility and reusability, among other advantages. In certain embodiments, the mask may simply retain a charge (i.e., a static charge) by applying friction to the mask material or through applying a recharging ionic spray to the mask material. A static charge may also be applied by subjecting the mask material to an electric hairdryer.

The mask may further power source can be configured to apply a charge to a fine wire mesh encompassing the mask surface. Alternatively, the mask surface can be impregnated with a particulate metal to distribute the charge evenly throughout the mask. It is contemplated herein that the impregnated metal can comprise a microparticles and nanoparticles to achieve its intended effect. It is further contemplated that the impregnated metal can be aluminum, copper, zinc, nickel, tin, gold, iron, platinum, and the like, as well as alloys thereof. Certain metals may also be employed, either in tandem with, or independent of, an applied electrical charge due to their anti-bacterial characteristics. For instance, copper, gold, and silver each demonstrate a pronounced antimicrobial effect that can allow particles filtered by the mask material and retained to be sterilized by contact with the mask material. In a similar manner, metal ions may be impregnated within the mask material by forming a salt solution and applying the salt solution to the mask material. Salts suitable for the purposes contemplated herein include, but are not limited to, sodium salts, calcium salts, magnesium salts, copper salts, iron salts, gold salts, silver salts, aluminum salts, gold salts, zinc salts, and combinations thereof. Metals can be included within masks and filtering materials as layers as described above to achieve a positively and negatively charged layer as described above. In certain embodiments, masks or filtering materials disclosed herein can comprise copper in a layer, or multiple layers

Methods for evaluating the effectiveness of masks for filtering viral particles are contemplated herein, and can include examining the viral load of a specific agent that passes from the external air space to an internal volume in the mask, considering the entirety of the mask as opposed to methods that assume no breach circumventing the mask material.

Methods to adapt single use respiratory masks to prolonged and extended use masks are contemplated herein, and provide masks able to conform to an individual wearer, offering an improved seal to restrict unfiltered air. Moreover, the methods and masks disclosed herein can provide a more comfortable product the user will be more likely to wear, more likely to wear properly, and more likely not to discard after a brief use. Improvements contemplated herein in turn reduce the chances that healthcare workers will become sick during treatment of patients, less likely to be carrier, less likely to transmit the infection to other patients. This reduction in infection further prevents reduction in the healthcare capacity and ability to treat patients within the healthcare system and become more likely to care for others and for a longer period if staying healthy, more likely to save others, and less likely to stay home due to fears of infection.

Methods to prolong the use of masks can lead to a need for sterilization and disinfecting the masks for reuse beyond single-use disposable masks. In certain embodiments, masks may be sterilized by exposure to heat and/or irradiated (e.g., with ultraviolet radiation). Accordingly, masks and mask materials contemplated herein may be UV and heat resistant to accommodate sterilization methods. For instance masks contemplated herein may be able to retain their characteristics (e.g., shape, flexibility, comfort) during elevated temperatures better than traditional N95 masks comprising melt-blown polypropylene material. Mask materials having reduced dependence on structural rigidity contemplated herein allow for improvements to the durability of the materials.

Each of the masks described above are also contemplated to further comprise a sterilization indicator, which can detect and signal an amount of heat or irradiation exposure that ensures completion of the sterilization procedure. The indicator is not limited to any particular display or implementation, and generally can be any method suitable to indicate to a user the effectiveness and progress of a sterilization procedure. In certain embodiments, the indicator can be reversible to accommodate multiple, and serial sterilization procedures after use. In certain embodiments, masks contemplated herein can comprise a plurality of indicators positioned about the mask to ensure adequate coverage, and provide multiple readings. In certain embodiments, the indicator can be a fragrance odor, or scent, or absence thereof that indicates the status of the sterilization process. The sterilization indicator is not limited to a particular form or arrangement, and generally can be any that are suitable to provide indication of the status of any sterilization process described herein. In certain embodiments, the sterilization indicator can comprise a first color and a second color, wherein the sterilization induces a color change from the first color to the second color. In certain aspects, the color change can be reversible during a time period after which the sterilization process has concluded, to allow the indicator to be effective following sequential uses. Alternatively, the indicator can be irreversible. In such aspects where the indicator is reversible, the indicator can be separately disposable from the article to allow the article to be reused, and potentially coupled to an unused indicator.

It is also contemplated herein that the color change between the first color and second color can be a gradient based on the dose or amount of exposure to a sterilization energy. In certain embodiments, the color change can occur at a minimum dose or a maximum dose, or one or more (e.g., 2, 3, 4, 5, 6, 7, etc.) intermediate doses to indicate progress in the sterilization process. Indicators comprising additional colors are also contemplated herein, and may include any number of colors such as a gradient of colors indicating the progress in sterilization. For example, the sterilization indicator can comprise a first color at a minimum dose, a second color at a maximum dose, and a gradient of colors between the first and second colors to indicate progress. In certain embodiments, the indicator can comprise a first color indicating the minimum dose has not been reached, a second color indicating the minimum dose has been received, and a third color indicating a maximum dose has been exceeded. The first and second colors can be reversible within the indicators disclosed herein, and the third color can be irreversible so as to indicate the article may have been damaged during the sterilization process.

Articles disclosed herein can comprise indicators in any arrangement. In certain embodiments, the indicator can be integrated within the article material such as within the filtering material of a mask as a colored spot. For instance, the fibers of a mask can be stained with an indicating material, to provide the color change as described above. Alternatively, the indicator can be a separate material integrated or attached to the article, and can be fashioned as a strip or dot, and attached by adhesive as a tape strip or spot.

Sterilization processes can comprise subjecting the article to a sterilization energy and/or sterilization composition for a sterilization period. The sterilization energy can be any form suitable to disinfect an article, and includes application of thermal energy and electromagnetic energy in any amount or degree. For instance, in certain embodiments, the sterilization energy can comprise a dose of UV radiation, UV-C radiation, microwave radiation, infrared radiation, or any combination thereof. Sterilization compositions contemplated herein can comprise a solid or solution containing chemical or biologic active ingredients. In certain embodiments, the sterilization composition can comprise a solution of ozone, hydrogen peroxide, or combinations thereof. The solution may also comprise compounds that specifically bind to a microorganism of interest. Such compounds can comprise small molecules that bind to external proteins and structures of the cells and antibodies.

Indicators contemplated herein can monitor and indicate the amount of a dose as described above as relative to a minimum, intermediate, and maximum dose, as a cumulative amount of the dose. In such embodiments, the indicator may be irreversible to account for previously applied doses. The indicator may also be reversible, and dependent on a time between radiation to reverse the indicating colors and negate the effects of the applied dose on the indicator.

In certain embodiments, the minimum dose can be defined as the minimum amount of sterilization energy required to kill a microorganism, or an amount of a microorganism. The microorganism can be any that are of interest to be sterilized during sterilization processes, and include bacteria, viruses, prions, yeasts, molds, fungi, and multicellular parasites such as mites.

Methods to adapt single use respiratory masks can further comprise the preparation of a gasket to insert between the mask edge and edge seal. Preparation of a gasket can comprise obtaining a gasket material as disclosed herein (e.g., a silicone sheet), a releaser such as an oil material, compression against the mask to shape the sheet against the mask, optionally marking the mask, and cutting the mask from the sheet of material.

Methods for safely removing and adjusting masks are also contemplated herein, and include pulling tabs present around the periphery of the mask (e.g., the mask edge), and particularly at the bridge of the nose. Conventional procedures for removing soiled masks include inserting the wearer's fingers under the straps to release the straps from the wearer's ears without contacting the filter portion of the mask which can be most heavily soiled. Masks comprising such tabs are also contemplated herein, and tables may be positioned projecting forward, substantially symmetric, between straps on sides, top and bottom, top only, bottom only, or any non-exclusive combination thereof. Methods to prepare masks comprising tabs are also contemplated herein. Generally, the tabs can be formed from any material, e.g., tape, adhered cellulosic material (e.g., cloth), a hook, or any combination thereof. The tabs may be built into the mask or added on.

Strap pads are also contemplated herein, and can generally be constructed from any material disclosed herein suitable to allow the strap to seat securely behind the ears of the wearer without resulting in discomfort during extended use. Suitable materials can comprise soft or cushioned materials described herein, compressible foam materials, or both.

Materials suitable for mask embodiments disclosed herein may be any of the following materials above may be non-porous, closed cell, UV resistant, ozone resistant, heat resistant, peroxide resistant, bleach resistant, alcohol resistant, heat resistant to a range but then degradable by heat after disposal, constructed from of filter material, or any combination thereof.

Methods for implementing the masks contemplated above comprising a self-adherent plastic can comprise wrapping a plastic comprising an integrated filter around the neck to create a seal on the face with air filter over the mouth or nose region to allow the patient to breathe through the filtered sealed over the face. It is normally considered dangerous to put such a plastic material over the face, but it this case it could be life saving, especially in times of severe shortages of PPE.

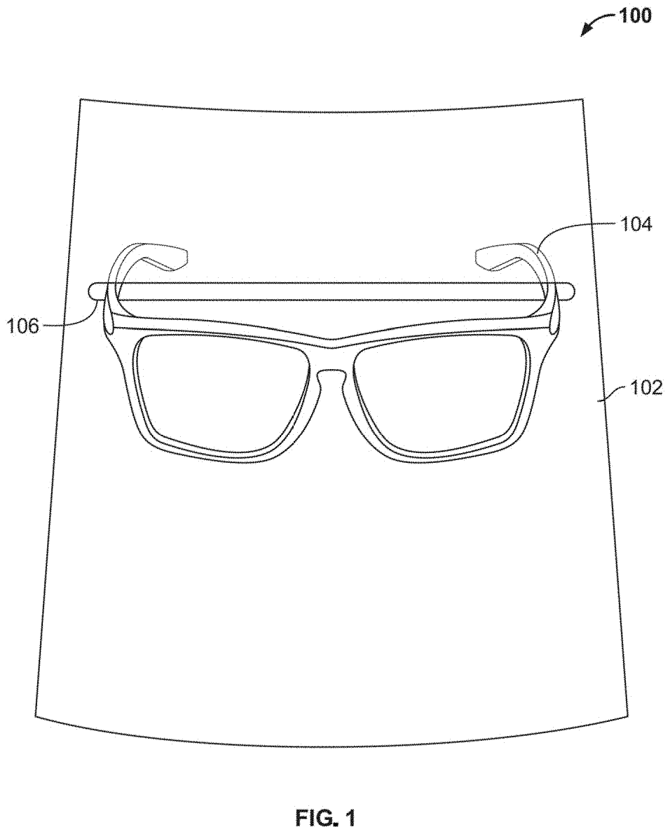

Certain embodiments are disclosed herein without a sealing arrangement. In further embodiments, respiratory masks may comprise a sheet of material with a slot allow the material to be supported by eyeglass frames. As shown in FIG. 1, respiratory mask 100 comprises rigid plastic sheet 102 comprising slit 106 in the material that extends longitudinally. Slit 106 allows displacement of a front and back edge of plastic sheet 102 forming the slit 106 and allowing plastic sheet 102 to seat onto eyeglasses frame 104.

A method of using a lamination sheet of clear plastic could be used with holes for the two side arms of a pair of prescription eyeglasses. Similarly a cough shield, such as a transparent cough shield with an aperture could be made that fits over a rigid or flexible suction device that may induce cough of dangerous respiratory particles. Alternatively, or additionally, certain embodiments may comprise an eyeglass gasket cut from a flexible sheet or block of material with a hole for the lens or lens of eyeglass and an attachment feature for restraining to the eyeglasses such as holes for the eyeglass arms.

Non-respiratory masks are also contemplated herein to protect bodily surfaces susceptible to infection by contact with unfiltered atmospheric air containing aerosolized infectious agents. For instance, eye protection and masks are contemplated in certain embodiments.

An embodiment of a respiratory hood comprising a filter can be constructed in a similar manner, by providing a vented inlet and outlet through a window in a transparent plastic and securing a filter to the vented inlet, outlet, or both. In certain embodiments, the hood can comprise a closed top and a seal around the body such as the neck at the bottom portion.

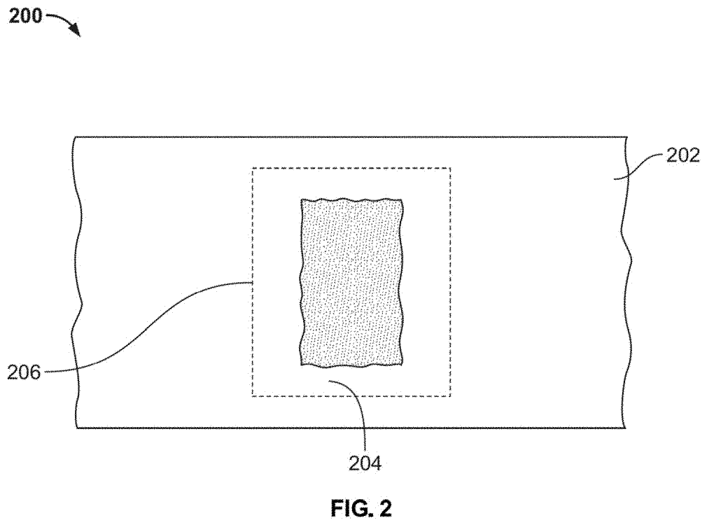

A representative embodiment of a respiratory hood contemplated herein is shown as FIG. 2. Respiratory hood 200 comprises plastic bag 202 and filter 204 disposed over a hole in the bag (not pictured). Filter 204 is attached to plastic bag 202 by an adhesive 206. Adhesive 206 can be any that is convenient to attach the filter to the plastic bag and form an air tight seal (e.g., packing tape).

Respiratory hoods enclosing a patient are also contemplated herein, which act to separate the treating staff from infected patients, and remove aerosolized infectious agents from the treatment facility. Such protective boxes can generally comprise a frame and a drape to allow the treating staff to administer treatment to the patient without risking exposure to aerosolized agents. Such protective boxes can be of particular benefit to procedures that are particularly prone to aerosolization of infectious agents, including intubations. Thus, as used herein, respiratory hoods can encompass an intubation box.

Intubation boxes disclosed herein can comprise any, all or none of the features described herein with respect to respiratory hoods. In certain aspects, respiratory hoods and intubation boxes can comprise a frame. The frame can be arranged generally in any manner that allows a drape to be supported over the patient and restrict airflow from the internal volume. In certain embodiments, the frame can be a wire or metal frame that seats on top of the patient as shown in the figure. In such embodiments, the treating staff can access the patient's face by inserting hands and instruments under the drape. Once treatment is completed, the drape can be replaced to return the protective box to prevent transmission of infectious agents throughout the room. In certain embodiments, the frame can be constructed of a disposable material and shipped flat. Examples of such materials include cardboard, and plastics that can be easily constructed on site. In certain embodiments, the frame will be disposable to allow quick and safe cleanup after the patient has been discharged. The frame therefore may be a box, wire cube, U-shaped wire, inflatable, or any combination thereof. The frame may be monolithic in certain embodiments. In certain aspects, the frame may have transparent windows embedded within the frame. The frame may comprise a drape, or a drape and filter. In such embodiments, the frame may further comprise a suction device such that the frame may be operated independently of environments lacking a source of negative airflow.

Methods of constructing respiratory hoods from components described herein are also contemplated. For instance, an intubation box can comprise folding box flaps inward to create a four-sided box frame with two opposing open ends. In such cases, the intubation box can have at least two sides that are transparent, and allowing access to the interior volume of the intubation box by means of the drape. Alternatively, the frame can be constructed from a U-shaped frame. In such aspects, the intubation box may have at least three sides that are transparent, and/or accessible via the drape.

Where the box is accessible by the drape, the intubation box and its internal volume will also be protected during access by the drape. Thus, the drape is not limited to any particular material, and generally can be any material or source capable to achieve this described purpose. In certain aspects, the drape can be a gusseted bag. In certain embodiments, the drape can have a closure to seal the drape to the frame, and can comprise overlapping walls or slats for sealing.

Certain specific embodiments of the frame and drape able to form the protective box are shown in the relevant figures, however, any suitable configurations to meet the needs discussed above are contemplated by the disclosure herein. In certain embodiments, the drape may encompass the frame on any number of sides. The frame may comprise a vacuum formed transparent shell, or alternatively comprise a foldable components. The frame may have a curved outer surface for strength or have sealed sides to a base and open ends that comprise drapes as transparent sealing features.

Protective boxes (e.g., intubation boxes and respiratory hoods) contemplated herein may also comprise attachments to assist in the removal of contaminated air from within the protective box to a secure location. For instance, negative suction can be provided to the protective boxes by a port configured to connect the internal volume of the box with a standard medical vacuum hosing present in hospitals. The negative suction can be continuous, and exceed the air exchange of negative pressure hospital rooms. In certain embodiments, the protective boxes may have multiple air exchanges. In this manner, contaminated air may be removed to a canister outside the room, and greatly reduce the amount of aerosolized infectious agent present, thereby reducing the risks of exposing medical staff. The amount of aerosolized agents can be reduced by 50%, 60%, 70%, 80%, or 90% relative to protective boxes which do not comprise a vacuum port to supply negative airflow to the protective box. The ports and hoses attached may also comprise filters.

The overall dimensions of protective boxes contemplated herein may be any that are suited for protecting the patient or healthcare worker during procedures such as intubation, and other described herein. In certain aspects, the enclosure may extend from a point beyond the top of a medical bed to any point of the patient. In certain embodiments, the enclosure may be configured to extend from at least the patient's head to beyond the chin, axilla, sternum, waist, knees, or toes of the patient, or beyond the end of the medical bed. Laterally, the enclosure may extend to encompass the width of the patients head beyond both ears, or beyond both shoulders, beyond a mattress, or beyond a medical bed including the rails. The enclosure may also have a height that extends from the top a patients face or head to a top surface of the mattress or a bottom surface of the mattress. Dimensions of protective boxes disclosed herein can be configured so as not to restrict application of chest compressions or other medical interventions to the rest of the body. In certain embodiments. The frame may be configured to attach to an IV pole or other fixture for stabilization. In certain aspects, the frame can comprise a ceiling attachment, or one or more IV pole attachments.

The drape may be any material that restricts airflow and the passage of an infectious agent of concern. In certain embodiments, it can be advantageous for the drape to be at least partially transparent to allow the treating staff to view their hands and instruments during treatment, and also provide comfort to the patient.

Certain non-limiting combinations of elements described herein are provided to illustrate exemplary embodiments. For instance, protective boxes contemplated herein can comprise a frame consisting of corrugated cardboard, a drape consisting of plastic. The drape can comprise a plastic bag or gusseted bag. The protective boxes can be disposable in certain embodiments. The frame may also comprise a copper surface, such as copper wire or tubing, and more broadly may comprise materials and properties that kill or prevent microbial growth. The frame as described herein may be an outside surface or inside surface of a box. The frame may be collapsible to a flat orientation for ease of shipping and storage, and configured to be subsequently unfolded during use.

Full body shields are also contemplated herein, and can generally comprise a large transparent sheet of any material as described herein (e.g., rigid or flexible plastic), and may generally be configured to form around the circumference of the wearer's body while allowing use of the wearer's arms through holes in the shield at the arm level. The shield can be fitted with a strap at the waist and the head to secure the shield without the use of the wearer's hands or relying on external supports that may be cumbersome during treatment of uncooperative or incapacitated patients. The body shield can cover upper body having a width greater than or equal to the width of a person's face, and/or greater than an adult body trunk width. Alternatively, the width can be greater than the upper body and less than elbow span. The width of the shield may be uniform throughout the length, or having a lower portion narrower than an upper portion to provide additional clearance for close proximity. For instance, a lower portion of the shield may be designed to allow space for the user's toes, feet, and knees, particularly when seated.

The body shield therefore may comprise a lower portion than a face protection portion having a width less than elbow-to-elbow width, or less than chest width allowing the user to reach around with both arms to the center of the opposing side of the shield. The body shield may further comprise a portion having a width less than the chest, less than the shoulders, less than the elbows, or any combination thereof. Thus, in certain embodiments, the body shield may have an insert that allows the user to conduct manual operations on the opposite side of the mask comfortably. Such aspects may be T-shaped or V-shaped, having an upper face portion wider than a lower body portion of the shield. Alternatively, the body shield may have an hourglass shape that comprises an upper face portion wider than an intermediate body portion, the intermediate body portion being narrower than a bottom body portion. The face protection portion of the shield can be wider than the head for protection of the patient, and from the patient, against direct coughing or breathing. Intermediate portions can constitute slots for the arms to extend through the shield, and may be horizontal, or diagonal at any angle above horizontal. In certain aspects the slots can be 10 degrees, 15 degrees, 20 degrees, 25 degrees, or 35 degrees from horizontal. The slots can extend from an upper portion to an intermediate portion of the shield. Space may also be provided beneath the shield portion for the user's knees while walking and moving the shield.

Hooks may also be provided in certain embodiments to stabilize the shield against the user. Wheels may be provided at the base of the shield to assist mobility. In certain embodiments, brakes to the wheels may also be provided, for instance at the foot or handle of the body shield. Any number of wheels that offer stability to the device while moving are contemplated herein. In certain aspects, the body shield may comprise two wheels, three wheels, four wheels or more than four wheels. Where wheels are present in the body shield, the wheels may be located at the base of the shield, and below any body portion of the shield. Wheels may further comprise locking mechanism. In certain embodiments, the wheels can be offset from the base of the shield so as to prevent interference with the feet of the user while walking and moving the shield. It is also contemplated herein that body shields of the present invention can comprise an adjustable height mechanism for adapting the shield height to the height of a range of users, and for different positions of both the patient and the healthcare professional, where each independently can be standing, sitting, or kneeling.

The shield portion may be transparent, in whole or in part. The shield portion may also be flat to accommodate simpler manufacturing, or alternatively curved for strength. Shields having contoured surfaces are also contemplated herein. Contoured surfaces are contemplated for enclosing the healthcare workers in a concave fashion. Alternatively, or additionally, convex countered shields are contemplated to provide enclosure and protection of a patient. Concave shields are also contemplated herein. Shields are contemplated herein for use by healthcare workers, but also contemplated for any use which requires close interaction between two people, for instance cutting hair, administering vaccinations or obtaining samples for COVID testing. Accordingly, the shields disclosed herein can be configured to be mobile in an outdoor setting for face to face interactions in a drive-up setting, where one party is within a vehicle and another is outside the vehicle.

The body shield may also comprise attachments for storage (e.g., shelves) and positioning, such as a stationary base and/or hanger. Body shields comprising L brackets are also contemplated herein. In certain embodiments the body shield can comprise 3 points or more bracing, at least one “bracket” point or at least two bracket points with shield forming other contact points and/or surface.

Body shields contemplated herein may also comprise antimicrobial surfaces. For instance, body shields can comprise copper supports within any portion of the shield, such as the face portion, upper portion, intermediate portion, lower portion, frame or base of the shield. The body shield may or may not comprise a frame surrounding a transparent shield. In certain embodiments, the body shield can have handles placed in any arrangement around the transparent shield, frame, or base, as described herein.

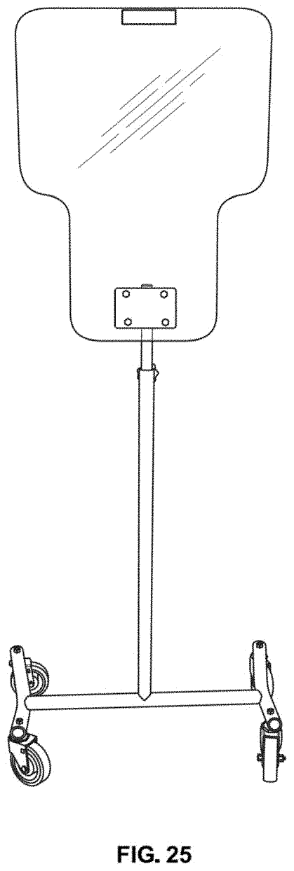

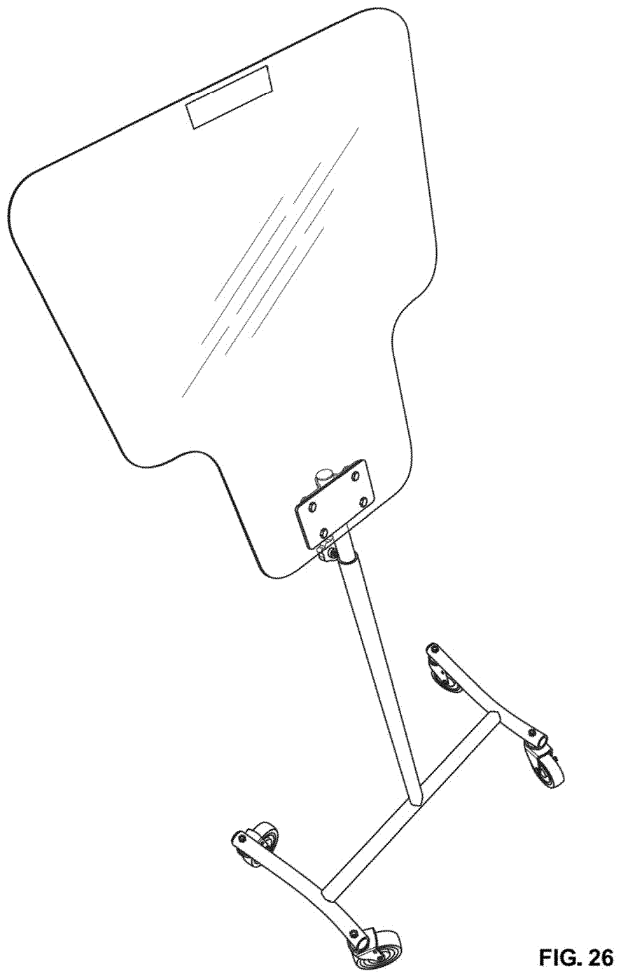

FIGS. 25-26 provides an exemplary embodiment of a body shield disclosed herein. As shown, the body shield can comprise a frame comprising a base and a vertical post. As shown, the base can comprise two parallel feet each supported by caster wheels at their endpoints and a crossbar connecting the parallel feet. The vertical post can comprise a fixed portion and a telescoping portion, the fixed portion extending vertically from the base a fixed distance in a range from about 2 to 4 feet, and the telescoping portion vertically extendible from the fixed portion a variable distance in a range from 1 to 3 feet. The vertical post can comprise a knob screw configured to secure the position of the telescoping portion by traversing the fixed portion and applying a force against the telescoping portion or traversing into the telescoping portion to restrict axial movement of the telescoping portion with respect to the fixed portion. The shield can be secured to the telescoping portion. Shield can comprise an upper portion that is wider than a lower portion.

As shown, the base portion can be asymmetric from front to back, to permit closer approach of the healthcare professional to the patient, displacing the shield toward the edge of the device while retaining the center of mass toward the center of the base portion to preserve stability of the shield. Shields and components may each have protected edges to prevent injury or damage to the surroundings during use. In certain aspects, the shield can have rounded edges, rounded corners, bumpers along the edges, or any combination thereof. Additionally, or alternatively, any or all components of the body shields disclosed herein independently can comprise antimicrobial surfaces such as copper or impregnated plastic materials to prevent contamination of the shield itself during use.

### Facial Tissue

Another embodiment of the invention is a reduced contamination tissue comprising a moisture/microbe impermeable substrate and backing, and an absorbent permeable layer to prevent transmission of microbes to the hands during use. This may be a facial tissue and may be provided in stacks and it may be provided in stacks like a box of facial tissue or in a roll form or in a roll form with perforations. The invention may have a softer pile side opposite the impermeable barrier or the barrier may be sandwiched between an absorbent or pile side that may have identical or different properties. The tissue may have a solution with cleaning properties and this may be on one or both sides. A removable layer may reveal and even activate a surface such as a cleaning surface by exposing a solution or compound that may be activated by exposure to the air. The tissues may be package in an antimicrobial or cleaning solution or a medication solution or antimicrobial solution so as to sanitize the hand or face after use, and may reside in a container with tissue disperser features such as folds that interlock or a dispenser opening.

I claim:

1. A face mask gasket kit for securing a perimeter of a filtering material to improve the filtering of air through the filtering material during breathing, the kit comprising a sealing gasket configured to seal gaps between the filtering material and a skin surface.

2. The kit of claim 1, wherein the sealing gasket comprises a paper, metal, tape, silicon material, rubber, plastic, foam, or combinations thereof.

3. The kit of claim 1, wherein the sealing gasket comprises a closed cell foam or a memory foam.

4. The kit of claim 1, wherein the sealing gasket comprises a gel, liquid, or viscous oil.

5. The kit of claim 1, wherein the sealing gasket comprises:
an adhesive layer configured to adhere the gasket to the skin surface;
an adhesive layer configured to adhere the gasket to the filtering material; or
both.

6. The kit of claim 5, wherein each of the first and second adhesive layers are protected by a liner removably secured to each adhesive layer.

7. The kit of claim 1, wherein the kit consists of the sealing gasket.

8. The kit of claim 1, further comprising the filtering material.

9. The kit of claim 8, wherein the filtering material comprises a face mask.

10. The kit of claim 9, wherein the face mask is a surgical mask, an N95 certified mask, or a KN95 certified mask.

11. The kit of claim 9, wherein the sealing gasket is configured to receive pressure from the face mask and induce a force against the skin surface.

12. The kit of claim 9, wherein the gasket is configured to induce a force against the face mask and the skin surface.

13. A face mask for securing a perimeter of a filtering material to improve the filtering of air through the filtering material during breathing, the face mask comprising the filtering material and a sealing gasket configured to seal gaps between the filtering material and a skin surface, wherein the sealing gasket and the filtering material form a flat, unitary sheet.

14. A portable body shield comprising:
a frame configured to attach to the body to be shielded; and
a shield portion attached to a frame, wherein the shield portion allows the user conduct manual operations on the opposite side.

15. The portable body shield of claim 14, wherein the shield portion comprises an upper shield portion that is wider than a lower body portion.

16. The shield of claim 14, wherein the frame comprises a plurality of wheels configured to interface with a floor surface during use.

17. The shield of claim 14, wherein the shield portion has an hourglass shape.

18. The shield of claim 14, wherein the shield portion is at least partially transparent.

19. The shield of claim 14, further comprising copper supports, handles, or both.

20. A portable body shield comprising:
a frame comprising a base and a vertical post, wherein:
the base comprises two parallel feet each supported by caster wheels at their endpoints and a crossbar connecting the parallel feet; and
the vertical post comprises a fixed portion and a telescoping portion, the fixed portion extending vertically from the base a fixed distance in a range from about 2 to 4 feet, and the telescoping portion extendible from the fixed portion a variable distance in a range from 1 to 3 feet, the vertical post comprising a knob screw configured to secure the position of the telescoping portion by traversing the fixed portion and applying a force against the telescoping portion or traversing into the telescoping portion to restrict axial movement of the telescoping portion with respect to the fixed portion; and
a shield secured to the telescoping portion, wherein the shield comprises an upper portion that is wider than a lower portion.
